(19) United States
(12) Patent Application Publication
(10) Pub. No.: US 2025/0090659 A1
(43) Pub. Date: Mar. 20, 2025

(54) COMBINATION THERAPY WITH AN ANTI-CD19 ANTIBODY AND PARSACLISIB

(71) Applicant: Incyte Corporation, Wilmington, DE (US)

(72) Inventor: Peter Langmuir, Cherry Hill, NJ (US)

(21) Appl. No.: 18/818,948

(22) Filed: Aug. 29, 2024

Related U.S. Application Data
(63) Continuation of application No. 18/413,277, filed on Jan. 16, 2024, now abandoned.
Application No. 18/413,277 is a continuation of application No. 17/538,050, filed on Nov. 30, 2021, now abandoned.
Application No. 17/538,050 is a continuation-in-part of application No. PCT/US2021/012982, filed on Jan. 11, 2021.
(60) Provisional application No. 63/119,370, filed on Nov. 30, 2020.

(51) Int. Cl. A61K 39/395; A61K 31/519; A61K 39/00; A61P 35/00

(52) U.S. Cl. CPC A61K 39/3955; A61K 31/519; A61P 35/00; A61K 2039/505; A61K 2039/545

(57) ABSTRACT

The present disclosure describes a combination of an anti-CD19 antibody and parsaclisib for the treatment of non-Hodgkin lymphoma, chronic lymphocytic leukemia, and acute lymphoblastic leukemia.

### CROSS-REFERENCE TO RELATED APPLICATIONS

This application is a continuation of U.S. application Ser. No. 18/413,277, filed on Jan. 16, 2024, which is a continuation of U.S. application Ser. No. 17/538,050, filed on Nov. 30, 2021, which claims priority to International Appl. No. PCT/US2021/012982, filed Jan. 11, 2021, and U.S. Provisional Appl. No. 63/119,370, filed Nov. 30, 2020. The content of the prior applications are incorporated by reference herein in their entirety.

### SEQUENCE LISTING

This application contains a Sequence Listing that has been submitted electronically as an XML file named “20443-0713003_SL_ST26.XML.” The XML file, created on Aug. 28, 2024, is 11,982 bytes in size. The material in the XML file is hereby incorporated by reference in its entirety.

### BACKGROUND

Non-Hodgkin lymphoma is the most common hematologic malignancy in adults. The majority of non-Hodgkin lymphomas are of B-cell origin, with multiple different histologic subtypes that confer different clinical outcomes. Apart from the pathologic classification of non-Hodgkin lymphomas, they are also commonly categorized as indolent or aggressive lymphomas based on their clinical characteristics. Indolent lymphomas are slowly progressing and responsive to therapy but are generally considered to be not curable. Aggressive lymphomas are rapidly progressing but responsive to therapy and often curable.

Diffuse large B-cell lymphoma (DLBCL), an aggressive and the most common non-Hodgkin lymphoma, accounts for one-third of non-Hodgkin lymphomas. Patients with DLBCL usually present with rapidly enlarging lymphoid masses, often with symptoms both local and systemic, and fever, recurrent night sweats, and/or weight loss. Some patients also have extranodal manifestations (e.g., skin, liver, gastric, CNS, and others). The current standard of care for the treatment of newly diagnosed DLBCL patients with advanced-stage disease is the immune chemotherapy regimen R-CHOP consisting of the anti-CD20 monoclonal antibody rituximab (R) plus CHOP (cyclophosphamide, doxorubicin, vincristine, and prednisone) chemotherapy. Following first-line therapy with R-CHOP, about 30% to 40% of patients relapse or are refractory to R-CHOP and require further treatment. Relapsed patients and patients with disease that fails to respond to first-line therapy (refractory patients) have a poor prognosis.

In addition to non-Hodgkin lymphoma, there are several other B cell malignancies that result from dysregulation of B cells. Chronic lymphocytic leukemia is an adult leukemia caused by an abnormal accumulation of B lymphocytes. In chronic lymphocytic leukemia, the malignant lymphocytes may look normal and mature, but they are not able to cope effectively with infection. Chronic lymphocytic leukemia is the most common form of leukemia in adults. Another type of leukemia, acute lymphoblastic leukemia, is characterized by the overproduction and continuous multiplication of malignant and immature white blood cells (also known as lymphoblasts) in the bone marrow. Acute lymphoblastic leukemia is most common in childhood with a peak incidence of 4-5 years of age.

### SUMMARY

The disclosure features a method of treating a non-Hodgkin lymphoma, chronic lymphocytic leukemia, or acute lymphoblastic leukemia in a human subject in need thereof by administering to the human subject a therapeutically effective amount of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one, or a pharmaceutically acceptable salt thereof, and an antibody that binds to human CD19, wherein the antibody comprises a variable heavy (VH) domain comprising VH complementarity determining region (CDR)1, VH CDR2, and VH CDR3, wherein:
the VH CDR1 comprises the amino acid sequence SYVMH (SEQ ID NO:6);
the VH CDR2 comprises the amino acid sequence NPYNDG (SEQ ID NO:7); and
the VH CDR3 comprises the amino acid sequence GTYYYGTRVFDY (SEQ ID NO:8); and
wherein the antibody comprises a variable light (VL) domain comprising VL CDR1, VL CDR2, and VL CDR3, wherein:
the VL CDR1 comprises the amino acid sequence RSSKSLQNVNGNTYLY (SEQ ID NO: 9);
the VL CDR2 comprises the amino acid sequence RMSNLNS (SEQ ID NO:10); and
the VL CDR3 comprises the amino acid sequence MQHLEYPIT (SEQ ID NO:11).

In some embodiments, the VH domain of the antibody comprises the amino acid sequence EVQLVESGGGLVKPGGSLKLSCAASGYTFTSYVMHWVRQAPGKGLEWIGYINPYND GTKYNEKFQGRVTISSDKSISTAYMELSSLRSEDTAMYYCARGTYYYGTRVFDYWG QGTLVTVSS (SEQ ID NO:4) and the VL domain of the antibody comprises the amino acid sequence

(SEQ ID NO: 5)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In some embodiments, the antibody comprises a heavy chain and a light chain, and wherein the heavy chain comprises the amino acid sequence set forth in SEQ ID NO:2 and the light chain comprises the amino acid sequence set forth in SEQ ID NO:3.

In some embodiments, the human subject has a non-Hodgkin lymphoma (e.g., relapsed/refractory non-Hodgkin lymphoma).

In some embodiments, the non-Hodgkin lymphoma is diffuse large B-cell lymphoma (e.g., relapsed/refractory diffuse large B-cell lymphoma).

In some embodiments, the non-Hodgkin lymphoma is follicular lymphoma (e.g., relapsed/refractory follicular lymphoma).

In some embodiments, the non-Hodgkin lymphoma is small lymphocytic lymphoma (e.g., relapsed/refractory small lymphocytic lymphoma).

In some embodiments, the non-Hodgkin lymphoma is mucosa-associated lymphoid tissue lymphoma (e.g., relapsed/refractory mucosa-associated lymphoid tissue lymphoma).

In some embodiments, the non-Hodgkin lymphoma is marginal zone lymphoma (e.g., relapsed/refractory marginal zone lymphoma).

In some embodiments, the non-Hodgkin lymphoma is Burkitt's lymphoma (e.g., relapsed/refractory Burkitt's lymphoma).

In some embodiments, the non-Hodgkin lymphoma is mantle cell lymphoma (e.g., relapsed/refractory mantle cell lymphoma).

In some embodiments, the human subject has chronic lymphocytic leukemia (e.g., relapsed/refractory chronic lymphocytic leukemia).

In some embodiments, the human subject has acute lymphoblastic leukemia (e.g., relapsed/refractory acute lymphoblastic leukemia).

In some embodiments, the antibody is administered intravenously. In some embodiments, the antibody is administered intravenously at a dose of 9 mg/kg or 12 mg/kg. In some embodiments, the antibody is administered intravenously at least once every two weeks at a dose of 9 mg/kg or 12 mg/kg. In some embodiments, the antibody is administered intravenously at a dose of 12 mg/kg according to the following schedule:
on days 1, 4, 8, 15, and 22 of a first 28-day cycle;
on days 1, 8, 15, and 22 of a second 28-day cycle;
on days 1, 8, 15, and 22 of a third 28-day cycle; and
on days 1 and 15 of a fourth 28-day cycle and on days 1 and 15 further 28-day cycles thereafter.

In some embodiments, 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally. In some embodiments, 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally at a dose of 1 mg, 2.5 mg, 5 mg, 10 mg, or 20 mg. In some embodiments, 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally once daily at a dose of 1 mg, 2.5 mg, 5 mg, 10 mg, or 20 mg.

In some embodiments, 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally once daily at a dose of 20 mg on days 1 to 56 and orally once daily at a dose of 2.5 mg thereafter.

In some embodiments, the antibody is administered intravenously at a dose of 12 mg/kg according to the following schedule:
on days 1, 4, 8, 15, and 22 of a first 28-day cycle;
on days 1, 8, 15, and 22 of a second 28-day cycle;
on days 1, 8, 15, and 22 of a third 28-day cycle; and
on days 1 and 15 of a fourth 28-day cycle and on days 1 and 15 further 28-day cycles thereafter, and
4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally once daily at a dose of 20 mg on days 1 to 56 and orally once daily at a dose of 2.5 mg thereafter.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a graph depicting synergy scores calculated with the Bliss synergy model for ADCC activity in JVM2 cells at different concentrations of tafasitamab (1:10 dilutions from 0.00001 μg/ml to 1 μg/ml) and parsaclisib (0, 0.3, 1, 3, 10, or 20 μM) in Experiment #1.

FIG. 1B is a graph depicting synergy scores calculated with the Bliss synergy model for ADCC activity in JVM2 cells at different concentrations of tafasitamab (1:10 dilutions from 0.00001 μg/ml to 1 μg/ml) and parsaclisib (0, 0.3, 1, 3, 10, or 20 μM) in Experiment #2.

FIG. 2 is a graph depicting the effects of the following treatments on tumor growth inhibition: (1) Vehicle; (2) Murine anti-CD19 antibody; (3) Parsaclisib (INCB050465); and (4) Murine anti-CD19 antibody+Parsaclisib (“Combo”).

FIG. 3 is a graph depicting the effects of the following treatments on tumor growth delay: (1) Vehicle; (2) Murine anti-CD19 antibody; (3) Parsaclisib (INCB050465); and (4) Murine anti-CD19 antibody+Parsaclisib (“Combo”).

### DETAILED DESCRIPTION

The combination of an anti-CD19 antibody and 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one, or a pharmaceutically acceptable salt thereof, described herein can be used to treat a non-Hodgkin lymphoma, chronic lymphocytic leukemia, or acute lymphoblastic leukemia.

### Anti-CD19 Antibodies

CD19 is broadly and homogeneously expressed across different B-cell derived blood cancers. CD19 is able to enhance B-cell receptor signaling, which is important for B-cell survival, and is therefore a therapeutic target for drugs aimed at treating B cell-related lymphomas and leukemias.

The amino acid sequence of the human CD19 protein is:

(SEQ ID NO: 1)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR.

Tafasitamab is an IgG1-G2/kappa Fc-engineered monoclonal antibody targeting human CD19. The constant Fc region of tafasitamab has been engineered to include the S239D and I332E (EU index) substitutions, which enhance the response of the immune system against cancer cells. The engineered Fc potentiates antibody-dependent cell-mediated cytotoxicity (ADCC) and antibody-dependent cellular phagocytosis (ADCP). Tafasitamab is described in U.S. Pat. No. 8,524,867, which is incorporated by reference in its entirety (in U.S. Pat. No. 8,524,867, the full heavy chain of tafasitamab is SEQ ID NO:87 and the full light chain of tafasitamab is SEQ ID NO:106).

The amino acid sequences of tafasitamab heavy and light chains are shown below. Complementarity-determining regions (CDRs) 1, 2, and 3 of the variable heavy (VH) domain and the variable light (VL) domain are shown in that order from N-terminus to the C-terminus of the mature VL and VH sequences and are both underlined and boldened.

Variable regions are underlined. An antibody consisting of the heavy chain (SEQ ID NO:2) and the light chain (SEQ ID NO:3) listed below is termed tafasitamab.
tafasitamab heavy chain:

(SEQ ID NO: 2)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

tafasitamab light chain:

(SEQ ID NO: 3)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

The variable heavy (VH) domain of tafasitamab has the following amino acid sequence:

(SEQ ID NO: 4)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

The variable light (VL) domain of tafasitamab has the following amino acid sequence:

(SEQ ID NO: 5)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK

The amino acid sequences of the VH CDRs of tafasitamab are listed below:

VH CDR1:
(SEQ ID NO: 6)
SYVMH;
VH CDR2:
(SEQ ID NO: 7)
NPYNDG;
and
VH CDR3:
(SEQ ID NO: 8)
GTYYYGTRVFDY.

The amino acid sequences of the VL CDRs of tafasitamab are listed below:

VL CDR1:
(SEQ ID NO: 9)
RSSKSLQNVNGNTYLY;
VL CDR2:
(SEQ ID NO: 10)
RMSNLNS;
and
VL CDR3:
(SEQ ID NO: 11)
MQHLEYPIT.

In certain embodiments, the anti-CD19 antibodies include a human heavy chain and light chain constant region. In certain embodiments, the heavy chain constant region comprises a CH1 domain and a hinge region. In some embodiments, the heavy chain constant region comprises a CH2 domain. In some embodiments, the heavy chain constant region comprises a CH3 domain. In some embodiments, the heavy chain constant region comprises CH1, CH2 and CH3 domains. If the heavy chain constant region includes substitutions, such substitutions modify the properties of the antibody (e.g., increase or decrease one or more of: Fc receptor binding, antibody glycosylation, the number of cysteine residues, effector cell function, or complement function). In certain embodiments, the antibody is an IgG antibody. In specific embodiments, the antibody is selected from the group consisting of IgG1, IgG2, IgG3, and IgG4.

Antibodies, such as tafasitamab, can be made, for example, by preparing and expressing synthetic genes that encode the recited amino acid sequences or by mutating human germline genes to provide a gene that encodes the recited amino acid sequences. Moreover, this antibody and other anti-CD19 antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16:237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., a human IgG1 constant region is mutated to include the S239D and/or I332E substitutions. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-CD19 antibodies can be in the form of full length antibodies, or in the form of low molecular weight forms (e.g., biologically active antibody fragments or minibodies) of the anti-CD19 antibodies, e.g., Fab, Fab′, F(ab′)₂, Fv, Fd, dAb, scFv, and sc(Fv)2. Other anti-CD19 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-CD19 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-CD19 antibody or antigen-binding fragment thereof comprising at least one deamidation site, wherein the pH of the composition is from about 5.0 to about 6.5, such that, e.g., at least about 90% of the anti-CD19 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising an anti-CD19 antibody or antigen-binding fragment thereof, means the presence of both the desired anti-CD19 antibody or antigen-binding fragment thereof and one or more acidic variants thereof. The acidic variants may comprise predominantly deamidated anti-CD19 antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

### Bispecific Antibodies

In certain embodiments, an anti-CD19 antibody or antigen-binding fragment thereof described herein is present in a bispecific antibody. Bispecific antibodies are antibodies that have binding specificities for at least two different epitopes. Exemplary bispecific antibodies may bind to two different epitopes of the CD19 protein. Other such antibodies may combine a CD19 binding site with a binding site for another protein. Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

In certain embodiments, an anti-CD19 antibody or antigen-binding fragment thereof described herein is present in a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹⁰Y, ¹³¹I) fluorescent substances, luminescent substances, haptens, enzymes, metal chelates, drugs, and toxins (e.g., calcheamicin, Pseudomonas exotoxin A, ricin (e.g. deglycosylated ricin A chain)).

In one embodiment, to improve the cytotoxic actions of anti-CD19 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or lgG4 isotypes).

In certain embodiments, an anti-CD19 antibody or antigen-binding fragment thereof are modified with a moiety that improves its stabilization and/or retention in circulation, e.g., in blood, serum, or other tissues, e.g., by at least 1.5, 2, 5, 10, or 50 fold. For example, the anti-CD19 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CD19 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CD19 antibody (e.g., tafasitamab) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

An anti-CD19 antibody or antigen-binding fragment thereof described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The anti-CD19 antibody or antigen-binding fragment thereof can be administered to a subject, e.g., a subject in need thereof, for example, a human subject, by a variety of methods.

For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody or antigen-binding fragment thereof can also be tailored for the individual case, e.g., by monitoring the subject, e.g., using tomographic imaging, e.g., to visualize a tumor.

The antibody or antigen-binding fragment thereof can be administered as a fixed dose, or in a mg/kg patient weight dose. The dose can also be chosen to reduce or avoid production of antibodies against the anti-CD19 antibody. Dosage regimens are adjusted to provide the desired response, e.g., a therapeutic response or a combinatorial therapeutic effect. Generally, doses of the anti-CD19 antibody can be used in order to provide a subject with the agent in bioavailable quantities. For example, doses in the range of about 9 mg/kg to about 12 mg/kg can be administered. In specific embodiments, a subject in need of treatment with an anti-CD19 antibody is administered the antibody at a dose of about 9 mg/kg or about 12 mg/kg. With respect to doses or dosages, the term “about” is intended to denote a range that is ±10% of a recited dose, such that, for example, a dose of about 3 mg/kg will be between 2.7 mg/kg and 3.3 mg/kg patient weight.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion. Exemplary fixed doses include about 675 mg, about 900 mg, about 1200 mg, and about 1800 mg. With respect to doses or dosages, the term “about” is intended to denote a range that is ±10% of a recited dose, such that, for example, a dose of about 375 mg will be between 337.5 mg and 412.5 mg.

An anti-CD19 antibody or antigen-binding fragment thereof dose can be administered, e.g., at a periodic interval over a period of time (a course of treatment) sufficient to encompass at least 2 doses, 3 doses, 5 doses, 10 doses, or more, e.g., once or twice daily, or about one to four times per week (e.g., at least twice per week), or weekly, biweekly (every two weeks), every three weeks, monthly, e.g., for between about 1 to 12 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

An exemplary dosing regimen comprises administration of an anti-CD19 antibody or antigen-binding fragment thereof at a fixed dose of about 675 mg once every 2 weeks. Another exemplary dosing regimen comprises administration of an anti-CD19 antibody or antigen-binding fragment thereof at a fixed dose of about 900 mg once every 2 weeks. Another exemplary dosing regimen comprises administration of an anti-CD19 antibody or antigen-binding fragment thereof at a fixed dose of about 1200 mg once every 2 weeks. Still another exemplary dosing regimen comprises administration of an anti-CD19 antibody or antigen-binding fragment thereof at a fixed dose of about 1800 mg once every 2 weeks.

An exemplary weight-based dosing regimen comprises administration of an anti-CD19 antibody or antigen-binding fragment thereof at a dosage of about 12 mg/kg at least twice per week, e.g., according to the following schedule:
on days 1, 4, 8, 15, and 22 of a first 28-day cycle;
on days 1, 8, 15, and 22 of a second 28-day cycle;
on days 1, 8, 15, and 22 of a third 28-day cycle; and
on days 1 and 15 of a fourth 28-day cycle and on days 1 and 15 further 28-day cycles thereafter.

A pharmaceutical composition may include a “therapeutically effective amount” of an anti-CD19 antibody or antigen-binding fragment thereof described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Parsaclisib

The present application provides methods of administering 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one (“parsaclisib” or “INCB050465”) or a pharmaceutically acceptable salt thereof in combination with an anti-CD19 antibody or antigen-binding fragment thereof.

In some embodiments, the compound administered is:
(S)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one;
(R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one;
(S)-4-(3-((R)-1-(4-amino-3-methyl-1H-pyrazolo[3,4-d]pyrimidin-1-yl)ethyl)-5-chloro-2-ethoxy-6-fluorophenyl)pyrrolidin-2-one; or
(R)-4-(3-((R)-1-(4-amino-3-methyl-1H-pyrazolo[3,4-d]pyrimidin-1-yl)ethyl)-5-chloro-2-ethoxy-6-fluorophenyl)pyrrolidin-2-one;
or a pharmaceutically acceptable salt thereof.

In some embodiments, the compound administered is a pharmaceutically acceptable salt of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one.

In some embodiments, the compound administered is selected from a pharmaceutically acceptable salt of:
(S)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one;
(R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one;
(S)-4-(3-((R)-1-(4-amino-3-methyl-1H-pyrazolo[3,4-d]pyrimidin-1-yl)ethyl)-5-chloro-2-ethoxy-6-fluorophenyl)pyrrolidin-2-one; or
(R)-4-(3-((R)-1-(4-amino-3-methyl-1H-pyrazolo[3,4-d]pyrimidin-1-yl)ethyl)-5-chloro-2-ethoxy-6-fluorophenyl)pyrrolidin-2-one.

In some embodiments, the compound administered is a pharmaceutically acceptable salt of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one.

In some embodiments, the compound administered is (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one hydrochloric acid salt.

In some embodiments, the salt is a 1:1 stoichiometric ratio of (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one to hydrochloric acid.

In some embodiments, the salt is crystalline.

In some embodiments, the hydrochloric acid salt of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one has: at least one, two, three, four or five XRPD peaks, in terms of 2-theta, selected from about 11.3°, about 16.4°, about 21.0°, about 23.0°, about 28.1°, about 31.2°, and about 32.8°; at least two XRPD peaks, in terms of 2-theta, selected from about 11.3°, about 16.4°, about 21.0°, about 23.0°, about 28.1°, about 31.2°, and about 32.8°; at least three XRPD peaks, in terms of 2-theta, selected from about 11.3°, about 16.4°, about 21.0°, about 23.0°, about 28.1°, about 31.2°, and about 32.8°;. In some embodiments, the hydrochloric acid salt of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one has at least four XRPD peaks, in terms of 2-theta, selected from about 11.3°, about 16.4°, about 21.0°, about 23.0°, about 28.1°, about 31.2°, and about 32.8°. In some embodiments, the hydrochloric acid salt of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one has all of the listed XRPD peaks, in terms of 2-theta, at about 11.3°, about 16.4°, about 21.0°, about 23.0°, about 28.1°, about 31.2°, and about 32.8°. In some embodiments, the hydrochloric acid salt of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one has a DSC thermogram having an endothermic peak at about 207° C.

Compounds described herein, including salts thereof, can be prepared using known organic synthesis techniques and can be synthesized according to any of numerous possible synthetic routes. In some embodiments, the compounds can be prepared as described in U.S. Pat. Nos. 9,199,982, 9,932,341, and 10,336,759, each of which is incorporated herein by reference in its entirety.

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety) and normal phase silica chromatography.

In some embodiments, the salts and compounds described herein are substantially isolated. By “substantially isolated” is meant that the salt or compound is at least partially or substantially separated from the environment in which it was formed or detected. Partial separation can include, for example, a composition enriched in the salts described herein. Substantial separation can include compositions containing at least about 50%, at least about 60%, at least about 70%, at least about 80%, at least about 90%, at least about 95%, at least about 97%, or at least about 99% by weight of the salts described herein, or salt thereof. Methods for isolating compounds and their salts are routine in the art.

### Labeled Compounds and Assay Methods

The methods of the present disclosure further includes the use of isotopically-labeled parsaclisib or a pharmaceutically acceptable salt thereof. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of an alkyl group of a compound described herein can be optionally substituted with deuterium atoms, such as —CD₃ being substituted for —CH₃).

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of the compounds described herein, are optionally replaced by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro PI3K labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

### Parsaclisib Pharmaceutical Formulations and Administration

Parsaclisib or a pharmaceutically acceptable salt thereof can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, parsaclisib or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl-and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration.

In some embodiments, the methods provided herein comprise:
i) administering to the human subject parsaclisib or a pharmaceutically acceptable salt thereof in a first dosage that is about 3 mg/day to about 20 mg/day for a first period of time of about 2 weeks to about 12 weeks; and
ii) administering to the human subject parsaclisib or a pharmaceutically acceptable salt thereof in a second dosage that is:
(a) about 2.5 mg/day or less; or
(b) about 50 mg/week or less;
and wherein the second dosage is administered for a second period of time that occurs after the first period of time.

In some embodiments, each of the first dosages is administered as a single, once daily dosage.

In some embodiments, each of the first dosages is administered as a single, once daily oral dosage.

In some embodiments, the first dosage is about 20 mg/day.

In some embodiments, the first dosage is about 20 mg/day and is administered as a single, once daily dosage.

In some embodiments, the first dosage is about 20 mg/day and is administered as a single, once daily oral dosage.

In some embodiments, the first dosage is about 10 mg/day.

In some embodiments, the first dosage is about 10 mg/day and is administered as a single, once daily dosage.

In some embodiments, the first dosage is about 10 mg/day and is administered as a single, once daily oral dosage.

In some embodiments, the first dosage is about 5 mg/day.

In some embodiments, the first dosage is about 5 mg/day and is administered as a single, once daily dosage.

In some embodiments, the first dosage is about 5 mg/day and is administered as a single, once daily oral dosage.

In some embodiments, the first period of time is about 3 weeks to about 11 weeks, for example, about 8 weeks to about 12 weeks, about 4 weeks to about 10 weeks, about 5 weeks to about 9 weeks, or about 8 weeks to about 9 weeks. In some embodiments, the first period of time is about 2 weeks, about 3 weeks, about 4 weeks, about 5 weeks, about 6 weeks, about 7 weeks, about 8 weeks, about 9 weeks, about 10 weeks, about 11 weeks, or about 12 weeks.

In some embodiments, the first period of time is about 8 weeks to about 9 weeks.

In some embodiments, the first period of time is about 8 weeks (or 56 days).

In some embodiments, the first period of time is about 9 weeks.

In some embodiments, the first dosage is reduced during the first period of time.

In some embodiments, each of the second dosages is administered as a single, once daily dosage.

In some embodiments, each of the second dosages is administered as a single, once weekly dosage.

In some embodiments, each of the second dosages is administered as a single, once daily oral dosage.

In some embodiments, each of the second dosages is administered as a single, once weekly oral dosage.

In some embodiments, the second dosage is about 5.0 mg/day or less, for example, about 5.0 mg/day, about 4.0 mg/day, about 3.0 mg/day, about 2.5 mg/day, about 2.0 mg/day, about 1.75 mg/day about 1.5 mg/day, about 1.25 mg/day, or about 1.0 mg/day.

In some embodiments, the second dosage is about 2.5 mg/day to about 7.5 mg/day.

In some embodiments, the second dosage is about 3.0 mg/day to about 7.0 mg/day.

In some embodiments, the second dosage is about 4.0 mg/day to about 6.0 mg/day.

In some embodiments, the second dosage is about 2.5 mg/day.

In some embodiments, the second dosage is about 2.5 mg/day and is administered as a single, once daily dosage.

In some embodiments, the second dosage is about 2.5 mg/day and is administered as a single, once daily oral dosage.

As used herein, “about” when referring to a measurable value such as an amount, a dosage, a temporal duration, and the like, is meant to encompass variations of ±10%.

### Indications

An anti-CD19 antibody or antigen-binding fragment thereof described herein (e.g., tafasitamab) and parsaclisib or a pharmaceutically acceptable salt thereof can be used in combination to treat a non-Hodgkin lymphoma in a human subject in need thereof. In some embodiments, the non-Hodgkin lymphoma is selected from the group consisting of follicular lymphoma, small lymphocytic lymphoma, mucosa-associated lymphoid tissue lymphoma, marginal zone lymphoma, diffuse large B cell lymphoma, Burkitt's lymphoma, and mantle cell lymphoma. In some embodiments, the non-Hodgkin lymphoma is relapsed/refractory diffuse large B-cell lymphoma.

Another aspect comprises a combination of an anti-CD19 antibody or antigen-binding fragment thereof described herein (e.g., tafasitamab) and parsaclisib or a pharmaceutically acceptable salt thereof for use in the treatment of a non-Hodgkin lymphoma. In some embodiments, the non-Hodgkin lymphoma is selected from the group consisting of follicular lymphoma, small lymphocytic lymphoma, mucosa-associated lymphoid tissue lymphoma, marginal zone lymphoma, diffuse large B cell lymphoma, Burkitt's lymphoma, and mantle cell lymphoma. In some embodiments, the non-Hodgkin lymphoma is relapsed/refractory diffuse large B-cell lymphoma.

Another aspect comprises a combination of an anti-CD19 antibody or antigen-binding fragment thereof described herein (e.g., tafasitamab) and parsaclisib or a pharmaceutically acceptable salt thereof in the manufacture of a medicament for treating a non-Hodgkin lymphoma. In some embodiments, the non-Hodgkin lymphoma is selected from the group consisting of follicular lymphoma, small lymphocytic lymphoma, mucosa-associated lymphoid tissue lymphoma, marginal zone lymphoma, diffuse large B cell lymphoma, Burkitt's lymphoma, and mantle cell lymphoma. In some embodiments, the non-Hodgkin lymphoma is relapsed/refractory diffuse large B-cell lymphoma.

An anti-CD19 antibody or antigen-binding fragment thereof described herein (e.g., tafasitamab) and parsaclisib or a pharmaceutically acceptable salt thereof can be used in combination to treat chronic lymphocytic leukemia in a human subject in need thereof.

Another aspect comprises a combination of an anti-CD19 antibody or antigen-binding fragment thereof described herein (e.g., tafasitamab) and parsaclisib or a pharmaceutically acceptable salt thereof for use in the treatment of chronic lymphocytic leukemia.

Another aspect comprises a combination of an anti-CD19 antibody or antigen-binding fragment thereof described herein (e.g., tafasitamab) and parsaclisib or a pharmaceutically acceptable salt thereof in the manufacture of a medicament for treating chronic lymphocytic leukemia.

An anti-CD19 antibody or antigen-binding fragment thereof described herein (e.g., tafasitamab) and parsaclisib or a pharmaceutically acceptable salt thereof can be used in combination to treat acute lymphoblastic leukemia in a human subject in need thereof.

Another aspect comprises a combination of an anti-CD19 antibody or antigen-binding fragment thereof described herein (e.g., tafasitamab) and parsaclisib or a pharmaceutically acceptable salt thereof for use in the treatment of acute lymphoblastic leukemia.

Another aspect comprises a combination of an anti-CD19 antibody or antigen-binding fragment thereof described herein (e.g., tafasitamab) and parsaclisib or a pharmaceutically acceptable salt thereof in the manufacture of a medicament for treating acute lymphoblastic leukemia.

In some embodiments, parsaclisib or a pharmaceutically acceptable salt thereof is administered prior to administration of the anti-CD19 antibody or antigen-binding fragment thereof.

In some embodiments, parsaclisib or a pharmaceutically acceptable salt thereof is administered after the administration of the anti-CD19 antibody or antigen-binding fragment thereof.

In some embodiments, the anti-CD19 antibody or antigen-binding fragment thereof and parsaclisib or a pharmaceutically acceptable salt thereof are administered simultaneously or together.

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Cytotoxicity of Combination of Tafasitamab and Parsaclisib

The cytotoxicity of tafasitamab and parsaclisib (INCB050465) alone and in combination is tested in a non-Hodgkin lymphoma (NHL), chronic lymphocytic leukemia (CLL), or acute lymphoblastic leukemia (ALL) cell line selected from any one of Ramos (Burkitt's Lymphoma), MEC-1 (CLL), HG-3 (CLL), CII (CLL), Su-DHL6 (DLBCL), U2932 (DLBCL), JVM-2 (Mantle Cell Lymphoma), and BALL-1 (ALL).

NHL, CLL, or ALL cells are pre-treated for 7 days with 0.3, 1, 3, or 10 μM of parsaclisib (parsaclisib alone group and tafasitamab and parsaclisib combination group). The tafasitamab alone group is pre-treated with DMSO for the same period of time.

NK cells are isolated from human primary PBMC from multiple donors using Easy Sep™ Human NK Cell Isolation Kit (STEMCELL Technologies) and frozen. NK cells are thawed and cultured with and without 2 μM of lenalidomide for 72 hours in medium supplemented with 2 IU/ml of IL-2.

After 7 days of parsaclisib treatment, NHL, CLL, or ALL cells are labeled with 10 μM of CFSE and co-cultured with NK cells pretreated with and without lenalidomide in the presence of various concentrations of tafasitamab for 4-16 hours. The effector to target cell ratio is 2:1. The cytotoxicity of tafasitamab alone is evaluated following the same procedure in NHL, CLL, or ALL cells that are not treated with parsaclisib. The doses of tafasitamab are 10-point 1:10 dilutions with the highest concentration starting at 15 μg/ml. After 4-16 hours incubation, cells are stained with DAPI or alternative cell viability dyes and measured by flow cytometry to evaluate cell toxicity. The data are presented as % dead cells and separated by NK donors.

The following controls are used: (i) NHL, CLL, or ALL cells+DMSO+NK cells, and (ii) NHL, CLL, or ALL cells+DMSO.

Combination index (CI) is calculated based on the isobol method (Ting-Chao Chou, 2010, Cancer Res. 70(2)). CI<1 is considered synergistically effective.

### Example 2: A Study of Tafasitamab in Combination with Parsaclisib in Subjects with Non-Hodgkin Lymphoma

This is an open-label, multicenter clinical study designed to assess tafasitamab-parsaclisib combination therapy in subjects with relapsed/refractory (R/R) diffuse large B-cell lymphoma (DLBCL).

Only biopsy-proven participants with DLBCL including one of the following diagnoses by 2016 WHO classification of lymphoid neoplasms are eligible to be included in the study: DLBCL, NOS including GBC type, ABC type.

Participants must have at least 1 bidimensionally measurable lesion. Participants must have at least 1 lesion that has a greatest transverse diameter of ≥1.5 cm and greatest perpendicular diameter of ≥1.0 cm at screening. The lesion must be confirmed to be PET-positive at the latest at the time of treatment.

Participants must have ECOG (Eastern Cooperative Oncology Group) performance status of 0 to 2.

Participants must have the following laboratory criteria at screening:
a. absolute neutrophil count (ANC)≥1.5×10⁹/L (unless secondary to bone marrow involvement by DLBCL as demonstrated by recent bone marrow aspiration and bone marrow biopsy, in this case absolute neutrophil count (ANC)≥1.0×10⁹/L).
b. Platelet count≥75×10⁹/L (unless secondary to bone marrow involvement by DLBCL as demonstrated by recent bone marrow aspiration and bone marrow biopsy, in this case Platelet count ≥ 50 × 10⁹/L).
c. Total serum bilirubin<1.5×upper limit of normal (ULN) unless secondary to Gilbert's syndrome or documented liver involvement by lymphoma. Participants with Gilbert's syndrome or documented liver involvement by lymphoma may be included if their total bilirubin is ≤5×ULN.
d. alanine aminotransferase (ALT), aspartate aminotransferase (AST), and alkaline phosphatase ≤3×ULN, or <5×ULN in cases of documented liver involvement.
e. Serum creatinine clearance must be ≥40 mL/minute either measured or calculated using a standard Cockcroft and Gault formula.

Participants must have relapsed or refractory DLBCL.

Participants must have received at least 1, but no more than 3, previous systemic therapy lines for the treatment of DLBCL. At least 1 previous therapy line must have included a CD20-targeted therapy (e.g., rituximab).

Participants must have failed autologous stem cell transplantation (ASCT) or in the opinion of the investigator currently have not been eligible for high-dose chemotherapy (HDC) with subsequent ASCT for participants with DLBCL.

Participants are excluded from the study if any of the following criteria apply:
1. Any other histological type of lymphoma according to WHO 2016 classification of lymphoid neoplasms, for example, primary mediastinal B-cell lymphoma (PMBL), Burkitt's lymphoma, B-cell lymphoma, unclassifiable, with features intermediate between DLBCL and classical Hodgkin lymphoma (grey-zone lymphoma); primary effusion lymphoma; primary cutaneous DLBCL, leg type; intravascular B cell lymphoma.
2. History of prior nonhematologic malignancy except for the following:
   a. Malignancy treated with curative intent and with no evidence of active disease present for more than 2 years before screening.
   b. Adequately treated lentigo maligna melanoma without current evidence of disease or adequately controlled non-melanomatous skin cancer.
   c. Adequately treated carcinoma in situ without current evidence of disease.
3. Congestive heart failure requiring use of ongoing maintenance therapy for life-threatening ventricular arrhythmias.
4. Participants with any of the following:
   a. Known positive test result for hepatitis C (HCV antibody serology testing) and a positive test for HCV RNA. Participants with positive serology must have been tested locally for HCV RNA and are eligible, in case of negative HCV RNA test results.
   b. Known positive test results for chronic HBV infection (defined by HBsAg positivity). Participants with occult or prior HBV infection (defined as negative HBsAg and positive total HBcAb) may be included if HBV DNA was undetectable (local test result), provided that they are willing to undergo ongoing DNA testing. Antiviral prophylaxis may be administered as per institutional guidelines. Participants who have protective titers of HBsAb after vaccination or prior but cured hepatitis B are eligible.
   c. Known seropositive for or history of active viral infection with HIV.
   d. Known active bacterial, viral, fungal, mycobacterial, or other infection at screening.
   e. Known CNS lymphoma involvement-present or past medical history.
   f. History or evidence of clinically significant cardiovascular, CNS and/or other systemic disease that would in the investigator's opinion preclude participation in the study or compromise the participant's ability to give informed consent.
   g. History or evidence of rare hereditary problems of galactose intolerance, Lapp lactase deficiency or glucose-galactose malabsorption.
   h. History or evidence of interstitial lung disease.
   i. Vaccination with live vaccine within 21 days prior to study treatment.
   j. Major surgery within up to 30 days prior to signing the informed consent form (ICF), unless the participant is recovered at the time of signing the ICF.
   k. Any anticancer and/or investigational therapy within 14 days prior to the start of Cycle 1.
   l. Gastrointestinal abnormalities including the inability to take oral study treatment, requiring IV alimentation, or prior surgical procedure affecting absorption.
   m. Pregnancy or lactation. Female participants who interrupt breastfeeding are able to be enrolled to the study. They must refrain from breastfeeding during the course of study and for 3 months after the last dose of study treatment.
5. Use or expected use during the study of any restricted medications, including potent CYP3A4 inhibitors or inducers within 14 days or 5 half-lives (whichever is longer) before the date of study treatment administration.
6. Participants who have:
   a. Not discontinued CD20-targeted therapy, chemotherapy, radiotherapy, investigational anticancer therapy, or other lymphoma-specific therapy within the 14 days prior to Day 1 dosing.
   b. In the opinion of the investigator, not recovered sufficiently from the adverse toxic effects of prior therapies.
   c. Previous treatment with CD19-targeted therapy (e.g., CD19-CAR-T therapies, other CD19 mAbs including bispecific and ADCs).
   d. Been previously treated with selective PI3Kδ or pan-PI3K inhibitors (e.g., idelalisib, copanlisib, duvelisib) and/or Bruton's tyrosine kinase inhibitors (e.g., ibrutinib).
   e. A history of hypersensitivity to compounds of similar biological or chemical composition to tafasitamab, IMiDs, and/or the excipients contained in the study treatment formulations (citric acid monohydrate, polysorbate 20, sodium citrate dehydrate and trehalose dihydrate).
   f. Undergone ASCT within the period ≤3 months before the signing of the ICF. Participants who have a more distant history of ASCT must exhibit full hematological recovery before enrolment into the study.
   g. Undergone previous allogenic stem cell transplantation.
   h. Concurrent treatment other anticancer or experimental treatments.

### Tafasitamab Regimen

For the first 3 cycles of the study, each cycle (Cycles 1-3) consists of a tafasitamab 12 mg/kg intravenous infusion on Day 1, Day 8, Day 15, and Day 22 of the cycle. Additionally, a loading dose is administered on Day 4 of Cycle 1. Thereafter, tafasitamab is administered on a bi-weekly basis with infusions on Days 1 and 15 of each repeated 28-day cycle until discontinuation. If the starting dose of tafasitamab needs to be reduced by 1 dose level, the lower dose levels is 9 mg/kg once weekly.

### Parsaclisib Regimen

Participants self-administer oral parsaclisib daily on Days 1 through 56 followed by a dose of 2.5 mg until discontinuation. The starting dose of parsaclisib is 20 mg once-daily on Days 1 through 56 followed by a 2.5 mg once-daily. If parsaclisib needs to be reduced by 1 dose level, the lower dose levels is as follows on Days 1 through 56, depending on what the current dose level is: 20 mg once-daily can be reduced to 10 mg once-daily or 10 mg once-daily can be reduced to 5 mg once-daily.

The endpoints of the study include:
a. incidence and severity of treatment-emergent adverse events (TEAEs) and incidence of dose-limiting toxicities (DLTs) with tafasitamab in combination parsaclisib,
b. pharmacokinetic (PK) analysis of tafasitamab as part of parsaclisib combination treatment,
c. objective response rate (ORR) as reported by investigator according to response criteria for lymphomas,
d. complete response rate (CRR) as reported by investigator according to response criteria for lymphomas,
e. progression-free survival (PFS) per investigator according to response criteria of lymphoma, and
f. Duration of response (DOR), defined as the time from first documented complete response (CR)/complete metabolic response (CMR) or partial response (PR)/partial metabolic response (PMR) until the date of first documented disease progression or death due to any cause, whichever occurs first, among participants who achieve a CR/CMR or PR/PMR as determined by an IRC according to the guidelines of the International Working Group, according to response criteria for lymphomas.

### Example 3: A Study of Tafasitamab in Combination with Parsaclisib in Subjects with Relapsed/Refractory Non-Hodgkin Lymphoma or Chronic Lymphocytic Leukemia

This is an open-label, multicenter clinical study designed to assess tafasitamab-parsaclisib combination therapy in subjects with relapsed/refractory (R/R) non-Hodgkin lymphoma or chronic lymphocytic leukemia.

Participants have R/R B-cell malignancies including R/R diffuse large B-cell lymphoma (DLBCL), R/R mantle cell lymphoma (MCL), R/R follicular lymphoma (FL), R/R marginal zone lymphoma (MZL), or R/R chronic lymphocytic leukemia (CLL)/small lymphocytic lymphoma (SLL).

Participants are eligible to be included in the study only if all of the following criteria apply:
1. Men and women aged ≥18 years at the time of consent.
2. Ability to comprehend and willingness to sign a written ICF and comply with all study visits and procedures.
3. Histologically confirmed R/R B-cell malignancy of the following types:
   a. Cohort 1: DLBCL not otherwise specified, T-cell/histiocyte-rich large B-cell lymphoma, Epstein-Barr virus-positive DLBCL of the elderly, Grade 3b FL, high-grade B-cell lymphoma with MYC and BCL2 and/or BCL6 rearrangements (double-hit or triple-hit lymphoma), histological transformation from an earlier diagnosis of low-grade lymphoma (such as FL, MZL, CLL) into DLBCL
   b. Cohort 2: MCL with documentation of either overexpression of cyclin D1 or t(11;14)
   c. Cohort 3: FL Grade 1, 2, and 3a
   d. Cohort 4: MZL, including extranodal, nodal, and splenic subtypes
   e. Cohort 5: CLL or SLL
4. Willingness to undergo biopsy requirements for the study, including an incisional, excisional, or core needle lymph node or tissue biopsy (or have archival lymph node or tissue from the most recent biopsy) and undergo bone marrow biopsy/aspirate collections as appropriate.
5. Received at least 2 prior systemic treatment regimens as follows:
   a. Cohorts 1 and 2 (DLBCL, MCL): Must have been previously treated with at least 1 prior chemoimmunotherapy regimen that included an anti-CD20 antibody. This includes treatments such as chemotherapy plus rituximab or obinutuzumab, with or without rituximab or obinutuzumab maintenance. Note: At least 6 doses of anti-CD20 chemoimmunotherapy must have been given in prior therapy.
   b. Cohorts 3 and 4 (FL, MZL): Must have been previously treated with at least 1 prior chemoimmunotherapy or immunotherapy regimen that included an anti-CD20 antibody. This includes treatments such as rituximab or obinutuzumab monotherapy or chemotherapy plus rituximab or obinutuzumab, with or without rituximab or obinutuzumab maintenance. Note: At least 6 doses of anti-CD20 immunotherapy must have been given in prior therapy.
   c. Cohort 5 (CLL/SLL): Must have been previously treated with at least 1 prior systemic therapy including a BTK inhibitor regimen or chemoimmunotherapy regimen that included an anti-CD20 antibody.
6. Relapsed, progressive, or refractory NHL or CLL:
   a. Relapsed: progressive disease (PD) after response of complete response (CR) to prior therapy.
   b. Progressive: PD after response of PR or stable disease to prior therapy.
   c. Refractory: achieved less than PR to the last prior therapy, or achieved a CR or PR that lasted <6 months before PD.
7. For Cohorts 1 to 4 and Cohort 5/SLL: radiographically measurable lymphadenopathy or extranodal lymphoid malignancy (defined as the presence of ≥1 lesion that measures >1.5 cm in the longest transverse diameter and ≥1.0 cm in the longest perpendicular diameter as assessed by CT or MRI.
8. ECOG performance status of 0 to 2.
9. Life expectancy >3 months.
10. LVEF (Left ventricular ejection fraction) ≥50%.
11. Laboratory results at screening as follows:
   a. Hemoglobin level ≥8 g/dL (unless secondary to bone marrow involvement by NHL/CLL, as demonstrated by recent bone marrow aspiration and bone marrow biopsy).
   b. ANC≥1.5×109/L (unless secondary to bone marrow involvement by NHL/CLL, as demonstrated by recent bone marrow aspiration and bone marrow biopsy).
   c. Platelet count≥75×109/L (unless secondary to bone marrow involvement by NHL/CLL, as demonstrated by recent bone marrow aspiration and bone marrow biopsy).
   d. Total serum bilirubin level≤1.5×ULN, or ≤5×ULN in cases of Gilbert syndrome or documented liver involvement by lymphoma.
   e. ALT, AST, and alkaline phosphatase level≤3×ULN, or ≤5×ULN in cases of documented liver involvement.
   f. Serum creatinine clearance ≥50 mL/min either measured or calculated using a standard Cockcroft and Gault formula (Cockcroft and Gault 1976).
12. Willingness to avoid pregnancy or fathering children based on the following criteria.
   a. Male participants with childbearing potential must agree to take appropriate precautions to avoid fathering children (with at least 99% certainty) from screening through 6 months after the last dose of study treatment and must refrain from donating sperm during this period.
   b. Female participants with childbearing potential must have a negative serum pregnancy test at screening and must agree to take appropriate precautions to avoid pregnancy (with at least 99% certainty) from screening through 90 days (or 180 days if required by local regulations) after the last dose of study treatment. They must also agree to regular urine pregnancy testing through the study treatment period. They must also refrain from breastfeeding and donating oocytes during the course of the study and for 3 months after the last dose of study treatment.
   c. Female participants without childbearing potential (ie, surgically sterile with a hysterectomy and/or bilateral oophorectomy OR≥12 months of amenorrhea and at least 50 years of age) are eligible. Female participants who have been amenorrheic for at least 12 months resulting from chemo/radiotherapy are considered of childbearing potential and should agree to use adequate contraceptive measures.

Participants are excluded from the study if any of the following criteria apply:
1. Any other histological type of lymphoma according to the WHO 2016 classification of lymphoid neoplasms, for example, primary mediastinal B-cell lymphoma, Burkitt lymphoma, B-cell lymphoma, unclassifiable, with features intermediate between DLBCL and classical Hodgkin lymphoma (gray zone lymphoma); primary effusion lymphoma; primary cutaneous DLBCL, leg type; intravascular B cell lymphoma.
2. History of or evidence of CNS lymphoma (primary and secondary).
3. Any anticancer and/or investigational therapy (e.g., chemotherapy, radiation therapy, surgery, immunotherapy, biologic therapy, hormonal therapy, or tumor embolization) within 30 days or 5 half-lives (whichever is greater) prior to the first dose of study treatment (C1D1).
4. Inadequate recovery (>Grade 1) from toxicity and/or complications from a major surgery before CID1.
5. Use or expected use during the study of any prohibited medications, including potent CYP3A4 inhibitors or inducers, within 14 days or 5 half-lives (whichever is longer) before C1D1.
6. Allogeneic stem cell transplantation within the past 6 months, or ASCT within 3 months before C1D1.
7. Previous treatment with CD19-targeted therapy (e.g., CD19-CAR-T therapies, other CD19 mAbs, including bispecific and antibody-drug conjugates) or PI3K inhibitors.
8. Treatment with corticosteroids at doses greater than physiologic doses (prednisone equivalent dose>10 mg/day) within 7 days of starting study treatment.
9. Clinically significant cardiac disease, including unstable angina, acute myocardial infarction, New York Heart Association Class II to IV congestive heart failure, uncontrolled arrhythmia, and/or cardiac conduction issues, within 6 months of C1D1.
10. Clinically significant concurrent, uncontrolled medical condition, including, but not limited to, renal, hepatic, hematologic, GI, endocrine, pulmonary, neurological, cerebral, or psychiatric disease.
11. Current or previous other malignancy within 3 years of study entry, except cured basal or squamous cell skin cancer, superficial bladder cancer, prostate intraepithelial neoplasm, or carcinoma in situ of the cervix.
12. Active graft-versus-host disease.
13. History of stroke or intracranial hemorrhage within 6 months of C1D1.
14. Chronic or current active infectious disease requiring systemic antibiotics, antifungal, or antiviral treatment within 30 days of C1D1.
15. Any of the following:
   a. Known positive test result for hepatitis C (HCV antibody serology testing) and a positive test result for HCV RNA. Participants with positive serology must have been tested locally for HCV RNA and are eligible in case of negative HCV RNA test results.
   b. Known positive test results for chronic HBV infection (defined by HBsAg positivity). Participants with occult or prior HBV infection (defined as negative HBsAg and positive total HBcAb) may be included if HBV DNA was undetectable (local test result), provided that they are willing to undergo ongoing DNA testing. Antiviral prophylaxis may be administered as per institutional guidelines. Participants who have protective titers of HBsAb after vaccination or prior but cured hepatitis B are eligible.
   c. Known seropositive for or history of active viral infection with HIV.
16. Inability to swallow and retain oral medication, malabsorption syndrome, disease significantly affecting GI function, total resection of the stomach or small bowel, ulcerative colitis, symptomatic inflammatory bowel disease, or partial or complete bowel obstruction.
17. History of hypersensitivity to compounds of similar biological or chemical composition to tafasitamab, parsaclisib, and/or the excipients contained in the study treatment formulations.
18. History of serious allergic reactions, including anaphylaxis and toxic epidermal necrolysis.
19. History or evidence of interstitial lung disease.
20. Exposure to vaccination with live vaccine within 30 days prior to C1D1, or anticipated need for such vaccination during treatment.
21. Currently pregnant or breastfeeding.
22. Any condition that would, in the investigator's judgment, interfere with full participation in the study, including administration of study treatment and attending required study visits; pose a significant risk to the participant; or interfere with interpretation of study data.

### Tafasitamab Regimen

For the first 3 cycles of the study, each cycle (Cycles 1-3) consists of a tafasitamab 12 mg/kg intravenous infusion on Day 1, Day 8, Day 15, and Day 22 of the cycle. Thereafter, tafasitamab is administered on a bi-weekly basis with infusions on Days 1 and 15 of each repeated 28-day cycle until discontinuation.

### Parsaclisib Regimen

Participants self-administer oral parsaclisib daily for 8 weeks followed by a dose of 2.5 mg until discontinuation. The starting dose of parsaclisib is 20 mg once-daily for 8 weeks followed by a 2.5 mg once-daily.

The endpoints of the study include:
a. Incidence and severity of TEAEs and incidence of DLTs.
b. ORR, defined as the percentage of participants having best response of CR/CMR or PR/PMR per investigator assessment.
c. PK parameters of tafasitamab when given in combination with parsaclisib. C_{trough} (i.e., predose), Cₘₐₓ, tₘₐₓ, Cₘᵢₙ, and AUCₜ will be summarized by descriptive statistics.
d. CRR, defined as the percentage of participants having best response of CR/CMR per investigator assessment.
e. DOR, defined as the time from the first documented CR/CMR or PR/PMR until the date of first documented disease progression or death due to any cause, whichever occurs first, among participants who achieve CR/CMR or PR/PMR.
f. PFS, defined as the time from the date of first dose of study treatment until the first documented disease progression, or death due to any cause, whichever occurs first.
g. OS, defined as the time from the date of first dose of study treatment until death due to any cause.
h. Percentage of participants who develop specific ADAs (antidrug antibodies) to tafasitamab.
i. Analysis of the dynamic composition of cytokines and immune cell populations and subpopulations in the peripheral blood by proteomics and flow cytometry.
j. Analysis of the tumor microenvironment using RNA expression signatures.
k. Analysis of CD19 and other mutations in peripheral blood using next generation sequencing (NGS).
l. Analysis of RNA expression profiles, RNA splicing patterns, and DNA mutations/indel/single nucleotide polymorphisms.
m. Evaluation of MRD (minimal residual disease) after response by polymerase chain reaction or NGS.

### Example 4: In Vitro Analysis of Cytotoxicity of Combination of Tafasitamab and Parsaclisib

The cytotoxicity of tafasitamab and parsaclisib (INCB050465) alone and in combination was tested in the following cells lines: MEC-1 (CLL), MEC-2 (CLL), JVM-2 (Mantle Cell Lymphoma), WSU-NHL, Pfeiffer, Su-DHL4, and Su-DHL6.

Target cells were pre-treated for 7 days with various doses of parsaclisib in the range of 0.3 to 20 μM (parsaclisib alone group and tafasitamab and parsaclisib combination group). The tafasitamab alone group was pre-treated with DMSO for the same period of time.

NK cells were isolated from human primary PBMC from multiple donors using EasySep™ Human NK Cell Isolation Kit (STEMCELL Technologies) and frozen. NK cells were thawed and cultured overnight in medium supplemented with 2 IU/ml of IL-2 (Peprotech) 24 hours before the ADCC assay.

The following controls were used in all experiments: (i) Target cells+NK cells without antibodies; (ii) Target cells+NK cells+huIgG1 isotype control antibody; and (iii) Target cells alone.

After 7 days of parsaclisib treatment, target cells were harvested and labeled with 0.1 μM of CellTrace™ Far Red Cell Proliferation dye (ThermoFisher C34564) and co-cultured with NK cells in the presence of various concentrations of tafasitamab for 4 hours. The effector to target cell ratio was 2:1. The proliferative inhibition effect on target cells caused by parsaclisib was included by adjusting the E: T ratio in inhibitor treated cells.

The cytotoxicity of tafasitamab alone was evaluated following the same procedure in cells that were treated with DMSO. The doses of tafasitamab were 10-point 1:10 dilutions with the highest concentration starting at 1 μg/ml. After 4 hours incubation, cells were stained with Zombie Violet fixable cell viability dye (Biolegend 423114) and measured by flow cytometry to evaluate cell toxicity. The data were presented as percentages of dead target cells minus the percentage of cell death from the “Target cells alone” well to subtract the background. NK cells used were from two healthy human donors.

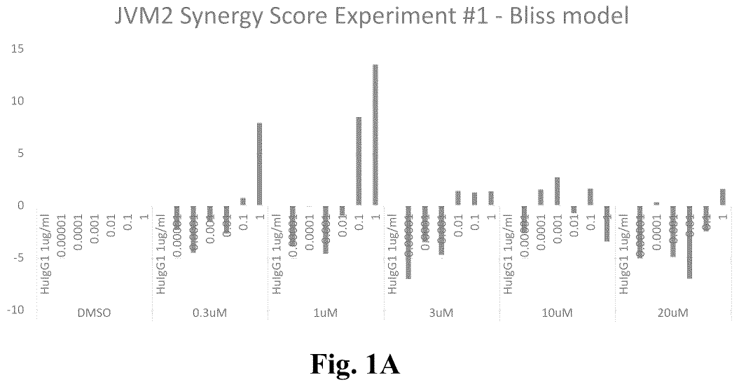

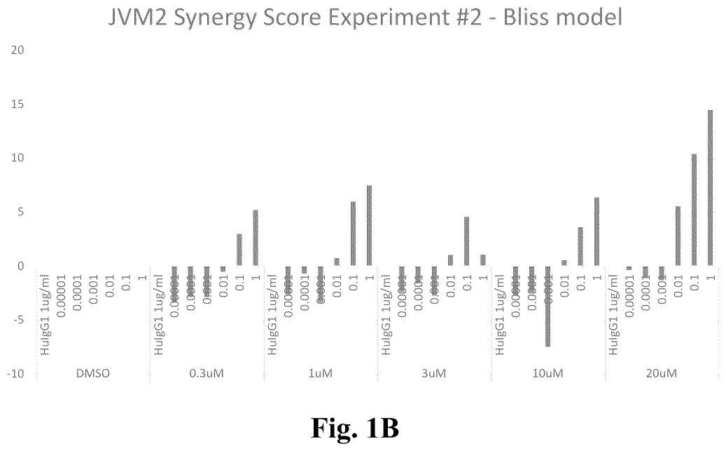

Tafasitamab displayed ADCC activity in a dose dependent manner in Mec1 and JVM2 cells. The combination of tafasitamab and parsaclisib showed further ADCC activity in addition to tafasitamab and parsaclisib treatments alone in JVM2 cells, but not in Mec1 cells. Data from two separately conducted experiments were analyzed to assess potential synergistic activity of tafasitamab and parsaclisib in JVM2 cells. In both experiments, selected doses of tafasitamab and parsaclisib exhibited synergistic effects in JVM2 cells, as indicated by Synergy scores>10. See FIGS. 1A-1B, assessing synergy with the Bliss synergy model in each of the experiments. Synergy scores were calculated using SynergyFinder 2.0 (https://synergyfinder.fimm.fi). A synergy score>10 is considered synergistically effective.

Besides ADCC cytotoxicity, effects of tafasitamab and parsaclisib on cell proliferation were also evaluated by treating target cells with matrix dosing of tafasitamab and parsaclisib in the absence of NK cells. After 24 hours, 48 hours, and 72 hours, proliferation was assessed by adding 100 ul of Cell titer Glo to wells containing 100 ul of treated cells in duplicates. The data was presented as RLUs, which is in proportion to cell proliferation. Pfeiffer and WSU-NHL cells were sensitive to parsaclisib treatment and its proliferation was inhibited by the treatment of parsaclisib in time and dose dependent manners. Mec2 and JVM2 cells were sensitive to tafasitamab treatment and its proliferation was inhibited by tafasitamab in time and dose dependent manners. SuDHL4 and SuDHL6 cells showed sensitivity to neither tafasitamab nor parsaclisib treatments. No synergistic effects for the combination of tafasitamab and parsaclisib were observed.

### Example 5: In Vivo Efficacy Study with Combination of Anti-CD19 Antibody and Parsaclisib in Mouse Lymphoma Model

Female Balb/c mice were subcutaneously inoculated with 5×10⁵ A20 murine lymphoma cells to form flank tumors. When tumors reached approximately 100 mm³, mice were randomized into 4 groups for dosing (N=10 mice/group). Dosing groups were as follows: (1) Vehicle (5% dimethyl acetimde+95% methylcellulose)-administered by mouth twice a day; (2) Murine anti-CD19 antibody (12014B35 from Absolute Antibody)—150 mg administered intraperitoneally once weekly; (3) Parsaclisib (INCB050465)—3 mg/kg administered by mouth twice a day; and (4) Murine anti-CD19 antibody+Parsaclisib.

Tumor volumes were measured twice weekly. The study concluded when mean tumor volumes of each group reached 2000 mm³. All doses were tolerated as assessed by body weights. Percent tumor growth inhibition (TGI %) was calculated by: [1-(mean tumor volume of test group)/(mean tumor volume of vehicle group)]×100. Tumor growth delay (TGD) was determined by the number of days needed (approximate) after dosing ceases for mean tumor volumes of dosed groups to reach the final mean tumor volume of the vehicle group.

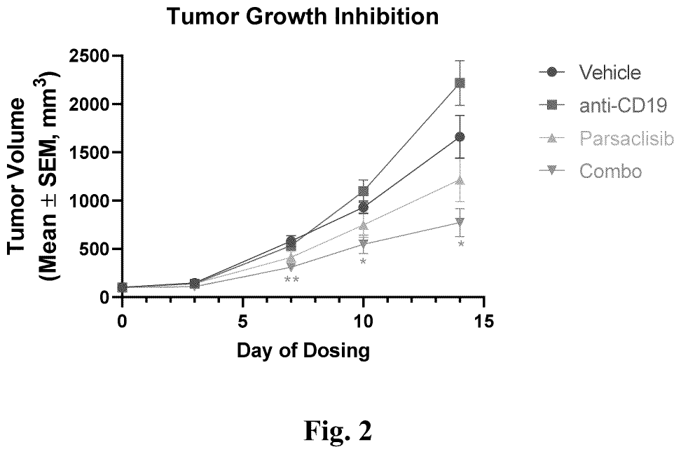

The effect of combination treatment with an anti-CD19 antibody and parsaclisib on TGI was greater than the effect that would have been expected based on single agent activities, indicating synergy associated with the combination. See FIG. 2. Only the anti-CD19 antibody+parsaclisib combination treatment group showed a statistically significant difference in TGI as compared to the Vehicle treated group. See Table 1. No statistical difference was found between the TGI from the combination group and the parsaclisib group.

TABLE 1
Effect of Treatments on Percent Tumor Growth Inhibition

| | | Statistical Significance |
|---|---|---|
| Treatment | TGI (%) | (p value vs. vehicle) |
| Vehicle | — | — |
| Anti-CD19 antibody | — | 0.333 |
| Parsaclisib | 26.6 | 0.523 |
| Combination | 53.5 | 0.020 |

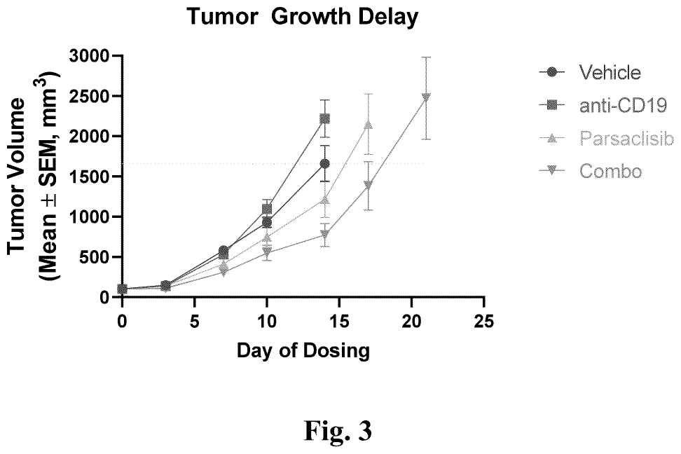

The effect of combination treatment with an anti-CD19 antibody and parsaclisib on TGD was greater than the effect that would have been expected based on single agent activities, indicating synergy associated with the combination. See FIG. 3 and Table 2.

TABLE 2
Effect of Treatments on Tumor Growth Delay

| Treatment | TGD (days) |
|---|---|
| Vehicle | — |
| Anti-CD19 antibody | — |
| Parsaclisib | 1.4 |
| Combination | 3.0 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a non-Hodgkin lymphoma, chronic lymphocytic leukemia, or acute lymphoblastic leukemia in a human subject in need thereof, the method comprising administering to the human subject a therapeutically effective amount of 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one, or a pharmaceutically acceptable salt thereof, and an antibody that binds to human CD19, wherein the antibody comprises a variable heavy (VH) domain comprising VH complementarity determining region (CDR)1, VH CDR2, and VH CDR3, wherein:
the VH CDR1 comprises the amino acid sequence SYVMH (SEQ ID NO:6);
the VH CDR2 comprises the amino acid sequence NPYNDG (SEQ ID NO:7); and
the VH CDR3 comprises the amino acid sequence GTYYYGTRVFDY (SEQ ID NO:8); and
wherein the antibody comprises a variable light (VL) domain comprising VL CDR1, VL CDR2, and VL CDR3, wherein:
the VL CDR1 comprises the amino acid sequence RSSKSLQNVNGNTYLY (SEQ ID NO:9);
the VL CDR2 comprises the amino acid sequence RMSNLNS (SEQ ID NO:10); and
the VL CDR3 comprises the amino acid sequence MQHLEYPIT (SEQ ID NO:11).

2. The method of claim 1, wherein the VH domain comprises the amino acid sequence EVOLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:4) and the VL domain comprises the amino acid sequence
(SEQ ID NO: 5)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

3. The method of claim 1, wherein the antibody comprises a heavy chain and a light chain, and wherein the heavy chain comprises the amino acid sequence set forth in SEQ ID NO: 2 and the light chain comprises the amino acid sequence set forth in SEQ ID NO:3.

4. The method of claim 1, wherein the human subject has a non-Hodgkin lymphoma.

5. The method claim 4, wherein the non-Hodgkin lymphoma is diffuse large B-cell lymphoma.

6. The method of claim 5, wherein the diffuse large B-cell lymphoma is relapsed/refractory diffuse large B-cell lymphoma.

7. The method claim 4, wherein the non-Hodgkin lymphoma is follicular lymphoma.

8. The method claim 4, wherein the non-Hodgkin lymphoma is small lymphocytic lymphoma.

9. The method claim 4, wherein the non-Hodgkin lymphoma is mucosa-associated lymphoid tissue lymphoma.

10. The method claim 4, wherein the non-Hodgkin lymphoma is marginal zone lymphoma.

11. The method claim 4, wherein the non-Hodgkin lymphoma is Burkitt's lymphoma.

12. The method claim 4, wherein the non-Hodgkin lymphoma is mantle cell lymphoma.

13. The method of claim 1, wherein the human subject has chronic lymphocytic leukemia.

14. The method of claim 1, wherein the human subject has acute lymphoblastic leukemia.

15. The method of claim 1, wherein the antibody is administered intravenously.

16. The method of claim 1, wherein the antibody is administered intravenously at a dose of 9 mg/kg or 12 mg/kg.

17. The method of claim 1, wherein the antibody is administered intravenously at least once every two weeks at a dose of 9 mg/kg or 12 mg/kg.

18. The method of claim 1, wherein the antibody is administered intravenously at a dose of 12 mg/kg according to the following schedule:
on days 1, 4, 8, 15, and 22 of a first 28-day cycle;
on days 1, 8, 15, and 22 of a second 28-day cycle;
on days 1, 8, 15, and 22 of a third 28-day cycle; and
on days 1 and 15 of a fourth 28-day cycle and on days 1 and 15 further 28-day cycles thereafter.

19. The method of claim 1, wherein 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally.

20. The method of claim 1, wherein 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally at a dose of 1 mg, 2.5 mg, 5 mg, 10 mg, or 20 mg.

21. The method of claim 1, wherein 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally once daily at a dose of 1 mg, 2.5 mg, 5 mg, 10 mg, or 20 mg.

22. The method of claim 1, wherein 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally once daily at a dose of 20 mg on days 1 to 56 and orally once daily at a dose of 2.5 mg thereafter.

23. The method of claim 1, wherein the antibody is administered intravenously at a dose of 12 mg/kg according to the following schedule:
on days 1, 4, 8, 15, and 22 of a first 28-day cycle;
on days 1, 8, 15, and 22 of a second 28-day cycle;
on days 1, 8, 15, and 22 of a third 28-day cycle; and
on days 1 and 15 of a fourth 28-day cycle and on days 1 and 15 further 28-day cycles thereafter, and
wherein 4-{3-[1-(4-amino-3-methyl-1H-pyrazolo[3,4-d]pyrimidin-1-yl)ethyl]-5-chloro-2-ethoxy-6-fluorophenyl}pyrrolidin-2-one or a pharmaceutically acceptable salt thereof is administered orally once daily at a dose of 20 mg on days 1 to 56 and orally once daily at a dose of 2.5 mg thereafter.


SEQUENCE LISTING (11 sequences)

SEQ ID NO: 1 - protein, 556 aa, Homo sapiens [sequence omitted]

SEQ ID NO: 2 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 5 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 6 - protein, 5 aa, synthetic construct
SYVMH

SEQ ID NO: 7 - protein, 6 aa, synthetic construct
NPYNDG

SEQ ID NO: 8 - protein, 12 aa, synthetic construct
GTYYYGTRVFDY

SEQ ID NO: 9 - protein, 16 aa, synthetic construct
RSSKSLQNVNGNTYLY

SEQ ID NO: 10 - protein, 7 aa, synthetic construct
RMSNLNS

SEQ ID NO: 11 - protein, 9 aa, synthetic construct
MQHLEYPIT